(19) United States
(12) Patent Application Publication
(10) Pub. No.: US 2015/0099960 A1
(43) Pub. Date: Apr. 9, 2015

(54) ULTRASONIC PROBE AND MEDICAL APPARATUS INCLUDING THE SAME

(71) Applicant: SAMSUNG ELECTRONICS CO., LTD., Suwon-si (KR)

(72) Inventors: Jae-young RYU, Suwon-si (KR); Young-hwan KIM, Hwaseong-si (KR); Jei-young LEE, Yongin-si (KR); Gwang-rok JUNG, Seoul (KR)

(73) Assignee: SAMSUNG ELECTRONICS CO., LTD., Suwon-si (KR)

(21) Appl. No.: 14/313,230

(22) Filed: Jun. 24, 2014

(30) Foreign Application Priority Data
Oct. 8, 2013 (KR) 10-2013-0120188

(51) Int. Cl. A61B 8/00; A61B 5/00; A61B 8/14; A61B 8/08

(52) U.S. Cl. CPC A61B 8/4444; A61B 8/485; A61B 5/0095; A61B 8/14; A61B 5/742
USPC 600/407; 600/459; 600/438; 600/443

(57) ABSTRACT

An ultrasonic probe includes a stimulation unit which stimulates an object so that particular waves are induced, a conversion unit which receives at least one of the particular waves and ultrasound including information about the particular waves, and a circuit board including a first circuit unit that drives the stimulation unit, and a second circuit unit that receives electrical signals corresponding to the ultrasound from the conversion unit.

### CROSS-REFERENCE TO RELATED APPLICATION

This application claims priority from Korean Patent Application No. 10-2013-0120188, filed on Oct. 8, 2013, in the Korean Intellectual Property Office, the disclosure of which is incorporated herein in its entirety by reference.

### BACKGROUND

1. Field

One or more exemplary embodiments relate to an ultrasonic probe and a medical apparatus including the same.

2. Description of the Related Art

An ultrasound diagnostic apparatus transmits ultrasound into an object of a living body, for example, a person or an animal, detects echo signals reflected from the object, displays a tomography image of tissue or tissues of the living body, and provides information used for diagnosis.

The ultrasound diagnostic apparatus includes an ultrasonic probe for transmitting the ultrasound into the object and receiving the echo signals from the object.

However, in some cases, it is difficult to accurately ascertain the mechanical characteristics and the like of tissue, because an ultrasound image of the related art may have low resolution.

### SUMMARY

Exemplary embodiments address at least the above problems and/or disadvantages and other disadvantages not described above. Also, the exemplary embodiments are not required to overcome the disadvantages described above, and may not overcome any of the problems described above.

One or more exemplary embodiments include an ultrasonic probe capable of stimulating an object and acquiring an image for the stimulated object.

According to one or more exemplary embodiments, an ultrasonic probe includes a stimulation unit which stimulates an object so that particular waves are induced, a conversion unit which receives at least one of the particular waves and ultrasound including information about the particular waves, and a circuit board including a first circuit unit that drives the stimulation unit, and a second circuit unit that receives an electrical signal corresponding to at least one of the particular waves and the ultrasound from the conversion unit.

The particular waves may include at least one of sound waves and shear waves.

Information about the particular waves may include at least one a displacement, a speed, and an intensity of the particular waves.

An arrangement of the stimulation unit and the conversion unit may correspond to that of the first circuit unit and the second circuit unit.

The stimulation unit may be disposed on an upper surface of the first circuit unit, and the conversion unit may be disposed on an upper surface of the second circuit unit.

The conversion unit may include a plurality of first conversion elements that convert ultrasound into an electrical signal or vice versa, and the plurality of first conversion elements may be arranged two-dimensionally.

At least one of the first circuit unit and the second circuit unit may include an application specific integrated circuit (ASIC).

The stimulation unit may provide light used to induce sound waves from the object.

The light may include pulse laser.

The stimulation unit may include a laser diode.

The second circuit unit may further include a driving circuit that drives the conversion unit.

The stimulation unit may include first and second stimulation units that are disposed separated from each other with the conversion unit interposed therebetween, and the first circuit unit may include a first sub-circuit unit that drives the first stimulation unit, and a second sub-circuit unit that drives the second stimulation unit.

The first and second sub-circuit units may independently drive the first and second stimulation units, respectively.

The first and second stimulation units may provide pressure waves used to induce shear waves from the object.

The pressure waves may include ultrasound.

A frequency of ultrasound transmitted by the first stimulation unit may be different from that of ultrasound transmitted by the second stimulation unit.

Each of the first and second stimulation units may include a plurality of second conversion elements that convert an electrical signal into ultrasound.

In each of the first and second stimulation units, the plurality of second conversion elements may be arranged one-dimensionally.

One of the first and second stimulation units may provide light, and the other may provide pressure waves.

The stimulation unit may further include third and fourth stimulation units that are disposed separated from each other with the conversion unit interposed therebetween, and the first circuit unit may further include a third sub-circuit unit that drives the third stimulation unit, and a fourth sub-circuit unit that drives the fourth stimulation unit.

At least one of the first and second stimulation units may be arranged together with at least one of the third and fourth stimulation units in a two-dimensional manner.

The first and second stimulation units may provide a first stimulus, and the third and fourth stimulation units may provide a second stimulus.

According to one or more exemplary embodiments, a medical apparatus includes the above-described ultrasonic probe, and a signal processor that processes a signal received from the ultrasonic probe to produce an image.

The medical apparatus may further include a display that displays the image.

### BRIEF DESCRIPTION OF THE DRAWINGS

The above and/or other aspects will be more apparent by describing certain exemplary embodiments, with reference to the accompanying drawings, in which:

FIG. 1 is a schematic diagram of an ultrasonic probe according to an exemplary embodiment;

FIGS. 2A and 2B illustrate an imaging portion of the ultrasonic probe of FIG. 1, according to an exemplary embodiment;

FIGS. 3, 4, 5, 6, and 7 illustrate examples of a conversion unit of the ultrasonic probe according to an exemplary embodiment;

FIG. 8 is a block diagram of a second circuit unit of the ultrasonic probe, according to an exemplary embodiment;

FIG. 9 illustrates a stimulation portion, according to an exemplary embodiment;

FIG. 10 illustrates a stimulation portion, according to an exemplary embodiment;

FIGS. 11, 12, and 13 are schematic perspective views of an ultrasonic probe having a plurality of stimulation portions, according to an exemplary embodiment;

FIG. 14 is a block diagram of a medical apparatus including an ultrasonic probe, according to an exemplary embodiment; and

FIGS. 15 and 16 are block diagrams of medical apparatuses according to exemplary embodiments.

### DETAILED DESCRIPTION

Exemplary embodiments are described in greater detail below with reference to the accompanying drawings.

In the following description, like drawing reference numerals are used for like elements, even in different drawings. The matters defined in the description, such as detailed construction and elements, are provided to assist in a comprehensive understanding of the exemplary embodiments. However, it is apparent that the exemplary embodiments can be practiced without those specifically defined matters. Also, well-known functions or constructions are not described in detail since they would obscure the description with unnecessary detail.

Expressions such as “at least one of,” when preceding a list of elements, modify the entire list of elements and do not modify the individual elements of the list.

Throughout the specification, a term ‘object’ may include a person, animal, or a part of a person or animal. For example, the object may include organs such as the liver, the heart, the womb, the brain, the breast, the abdominal region, etc., or a blood vessel. In the entire specification, a “user” may be a medical expert, such as a doctor, a nurse, a health care technician, or a medical imaging expert, or may be an engineer who manages medical appliances; however, an exemplary embodiment is not limited thereto.

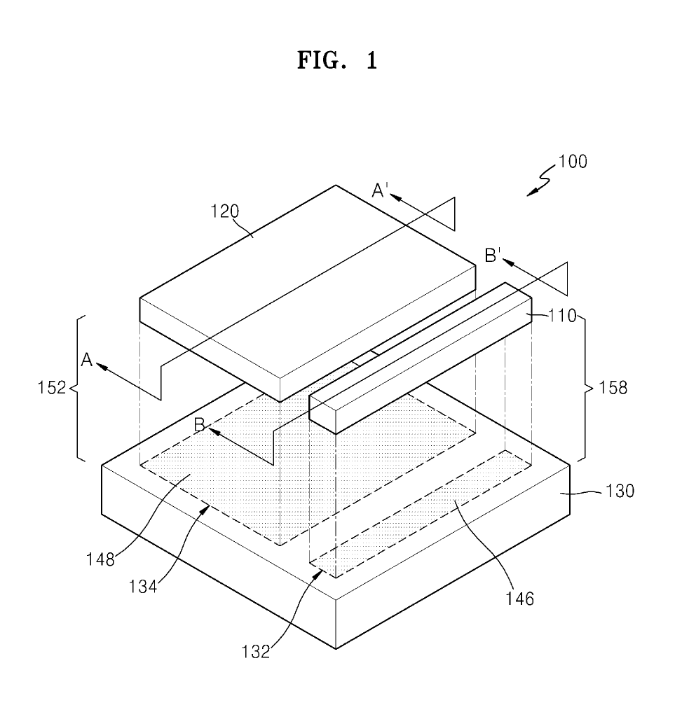

FIG. 1 is a schematic diagram of an ultrasonic probe 100 according to an exemplary embodiment. Referring to FIG. 1, the ultrasonic probe 100 includes a stimulation unit 110 for stimulating an object to induce particular waves, a conversion unit 120 for receiving ultrasound including information about the particular waves and converting the ultrasound into an electrical signal, and a circuit board 130 in which a first circuit unit 132 for driving the stimulation unit 110 and a second circuit unit 134 for receiving the electrical signal from the conversion unit 120 are formed on a substrate. The substrate may be formed of silicon (Si), ceramic, or a polymer-based material. The substrate may include an ultrasound-backing material that absorbs ultrasound. The first and second circuit units 132 and 134 may be implemented by using application specific integrated circuits (ASIC) and may be formed on a single substrate.

The stimulation unit 110 and the conversion unit 120 may be arranged in correspondence to an arrangement of the first and second circuit units 132 and 134. For example, the stimulation unit 110 may be disposed on an upper surface 146 of the first circuit unit 132, and the conversion unit 120 may be disposed on an upper surface 148 of the second circuit unit 134.

Since the first and second circuit units 132 and 134 are disposed on a single substrate, an object may be stimulated and an image thereof may be obtained, by the single ultrasonic probe 100. The stimulation unit 110 and the first circuit unit 132 together form a stimulation portion 158, and the conversion unit 120 and the second circuit unit 134 together form an imaging portion 152. The imaging portion and the stimulation portion of the ultrasonic probe 100 will now be described in greater detail.

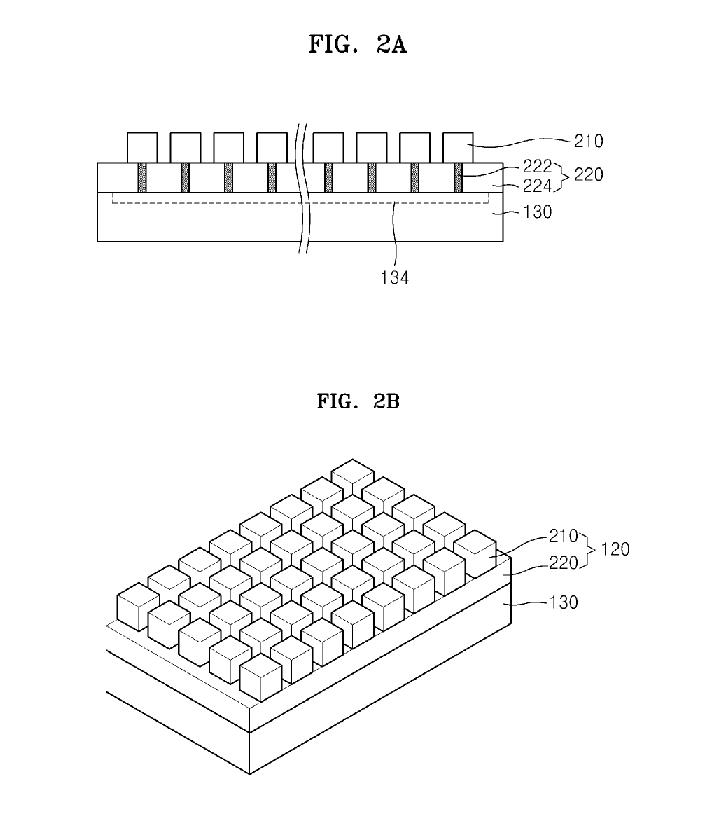

FIGS. 2A and 2B illustrate an imaging portion 152 of the ultrasonic probe 100, according to an exemplary embodiment. As illustrated in FIG. 2A, the conversion unit 120 of the imaging portion 152 may include a plurality of first conversion elements 210, which convert ultrasound into an electrical signal or vice versa. The first conversion elements 210 may be formed of a piezoelectric material that converts ultrasound into an electrical signal or vice versa due to vibrations. The first conversion elements 210 may be formed by splitting a piezoelectric material. For example, the first conversion elements 210 may be formed by dicing a piezoelectric material. However, the manufacture of the first conversion elements 210 is not limited to the aforementioned method, and thus, the first conversion elements 210 may be formed by using various other methods, such as, by pressing metal or a conductive material including metal. Examples of the piezoelectric material may include, but are not limited to, a piezoelectric ceramic, a single crystal material, and a composite piezoelectric material that cause a piezoelectric effect. The composite piezoelectric material is formed by compounding a polymer material and any of the aforementioned materials.

As illustrated in FIG. 2B, the first conversion elements 210 may be arranged in a two-dimensional (2D) array on a plane that is perpendicular to a direction in which ultrasound travels. The 2D conversion element array may be a linear array or a curved array. An array shape may vary according to a purpose or an application. The 2D conversion element array may acquire a more precise image, but the magnitude of a voltage applied to the first conversion elements 210 may be limited. For example, a voltage applied to each of the first conversion elements 210 may be 10V or less.

The conversion unit 120 may include a connection unit 220, which supports the first conversion elements 210 and electrically connects the first conversion elements 210 to the circuit board 130. In detail, the connection unit 220 may include an ultrasound-backing unit 222, which supports the first conversion elements 210 at a bottom side of the first conversion elements 210 and absorbs the ultrasound that is transmitted toward the bottom side of the first conversion elements 210 and that is not directly used in a test or a diagnosis, and a plurality of electrodes 224, which are disposed within the ultrasound-backing unit 222 and electrically connect the first conversion elements 210 to the second circuit unit 134.

The ultrasound-backing unit 222 may be formed of an attenuation material having a low acoustic impedance to absorb ultrasound, and the electrodes 224 may be formed of a conductive material. The electrodes 224 may be arranged separate from one another and may respectively electrically connect the first conversion elements 210 to the second circuit unit 134. The ultrasound-backing unit 222 may have a single-layered structure or may have a multi-layered structure in which a plurality of layers are coupled together horizontally. For example, the ultrasound-backing unit 222 may be formed so that a first ultrasound-backing layer is horizontally coupled to a second ultrasound-backing layer with electrodes 224 disposed therebetween. Although the single connection unit 220 supports the plurality of first conversion elements 210 in FIGS. 2A and 2B, exemplary embodiments are not limited thereto. Two or more connection units may support groups into which a plurality of first conversion elements are divided.

Although the described-above first conversion elements 210 of the conversion unit 120 are piezoelectric elements, exemplary embodiments are not limited thereto. For example, the conversion unit 120 may be a capacitive micromachined ultrasonic transducer (cMUT) that converts ultrasound into an electrical signal or vice versa due to a change in an electrostatic capacity, a magnetic micromachined ultrasonic transducer (mMUT) that converts ultrasound into an electrical signal or vice versa due to a change in a magnetic field, or an optical ultrasonic detector that converts ultrasound into an electrical signal or vice versa due to a change in optical characteristics.

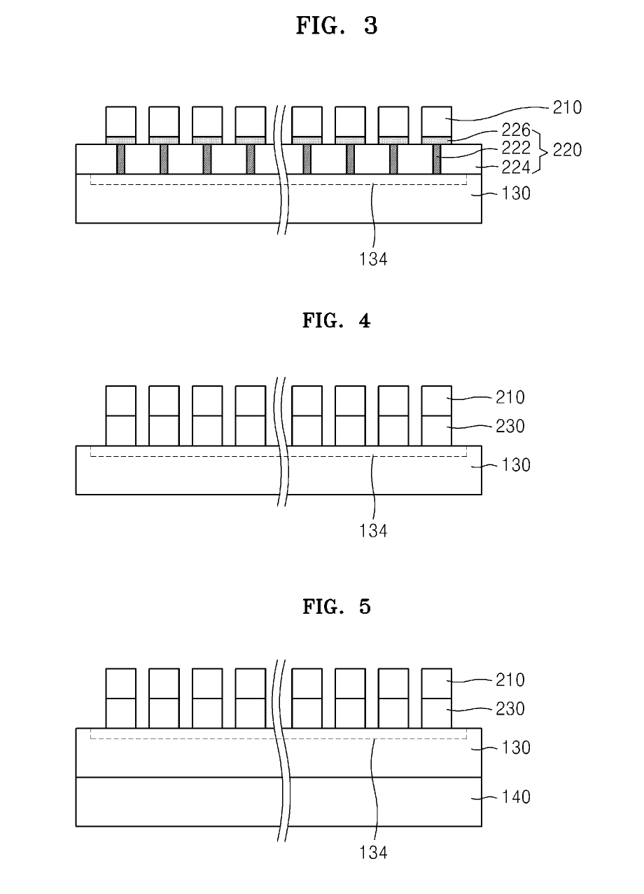

FIGS. 3 through 7 illustrate various examples of the conversion unit 120 that are applicable to an exemplary embodiment. As illustrated in FIG. 3, the connection unit 220 of the conversion unit 120 may further include electrodes 226 disposed on an upper surface of the ultrasound-backing unit 222. The electrodes 224 disposed on inner surfaces (i.e., lateral surfaces) of the ultrasound-backing unit 222 may be referred to as lateral electrodes, and the electrodes 226 disposed on an upper surface of the ultrasound-backing unit 222 may be referred to as upper electrodes. Due to further inclusion of the upper electrodes 226 in the connection unit 220 as illustrated In FIG. 3, the first conversion elements 210 and the second circuit unit 134 may be more firmly electrically connected to each other.

As illustrated in FIG. 4, the connection unit 220 may include by a plurality of connection elements 230. The connection elements 230 may support the first conversion elements 210, respectively, and may be separated from one another. Although the connection units 230 respectively support the first conversion elements 210 in FIG. 4, exemplary embodiments are not limited thereto. The connection elements 230 of FIG. 4 may be formed of a conductive material. Therefore, the connection elements 230 may support the first conversion elements 210 and at the same time may electrically connect the first conversion elements 210 to the second circuit unit 134.

Acoustic impedance of the connection elements 230 may be greater than acoustic impedance of the first conversion elements 210. Thus, ultrasound emitted toward the bottom side of the first conversion elements 210 may be reflected by the connection unit 220 toward the upper side of the first conversion elements 210. Consequently, emission efficiency of ultrasound may be effectively increased. The connection elements 230 may be formed of a conductive material having high acoustic impedance, such as, tungsten carbide or graphite. A bonding material (not shown) may be coated on the bottom surfaces of the connection elements 230 so that the connection elements 230 may be more easily bonded to the second circuit unit 134. The bonding material may be a conductive material such as tin (Sn), silver (Ag), or lead (Pb).

As illustrated in FIG. 5, an ultrasound-backing unit 140 may be further disposed on the bottom surface of the circuit board 130. Thus, ultrasound may be prevented from being transmitted into a region of the ultrasonic probe 100 that exists below the circuit board 130.

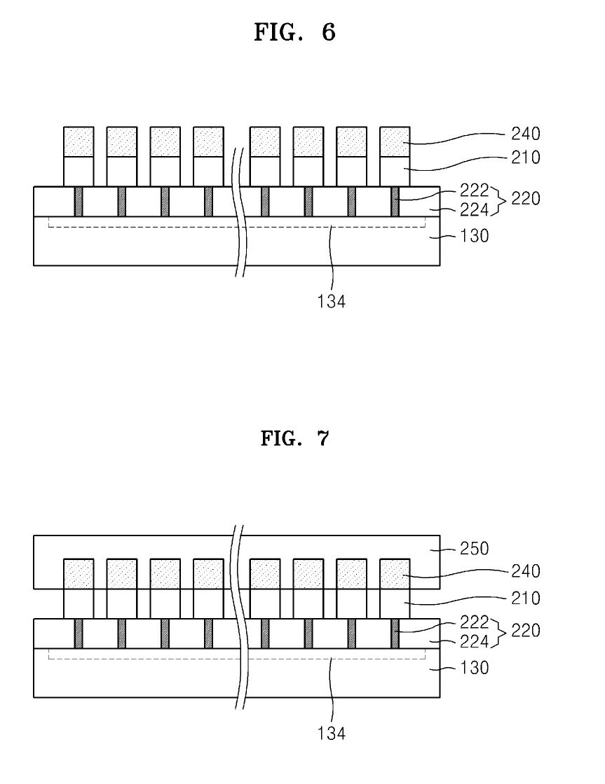

As illustrated in FIG. 6, the conversion unit 120 may further include a matching unit 240 for matching acoustic impedance of ultrasound generated by the first conversion elements 210 with acoustic impedance of an object. The matching unit 240 is disposed on the upper surfaces of the first conversion elements 210, and alters, in a stepwise manner, the acoustic impedance of the ultrasound generated by the first conversion elements 210, which causes the acoustic impedance of the ultrasound to be similar to the acoustic impedance of the object. In the present exemplary embodiment, the matching unit 240 has a single-layered structure. However, as another example, the matching unit 240 may have a multi-layered structure.

As illustrated in FIG. 7, the conversion unit 120 may further include an acoustic lens 250 for focusing ultrasound. The acoustic lens 250 is disposed on the upper surface of the first conversion elements 210, and functions to focus the ultrasound generated by the first conversion elements 210. The acoustic lens 250 may be formed of a material such as a silicon rubber having acoustic impedance that is similar to the acoustic impedance of the object. A central portion of the acoustic lens 250 may be convex or flat. The acoustic lens 250 may have various shapes according to various designs and applications.

The second circuit unit 134 may receive an electrical signal from the conversion unit 120 and/or may drive the conversion unit 120.

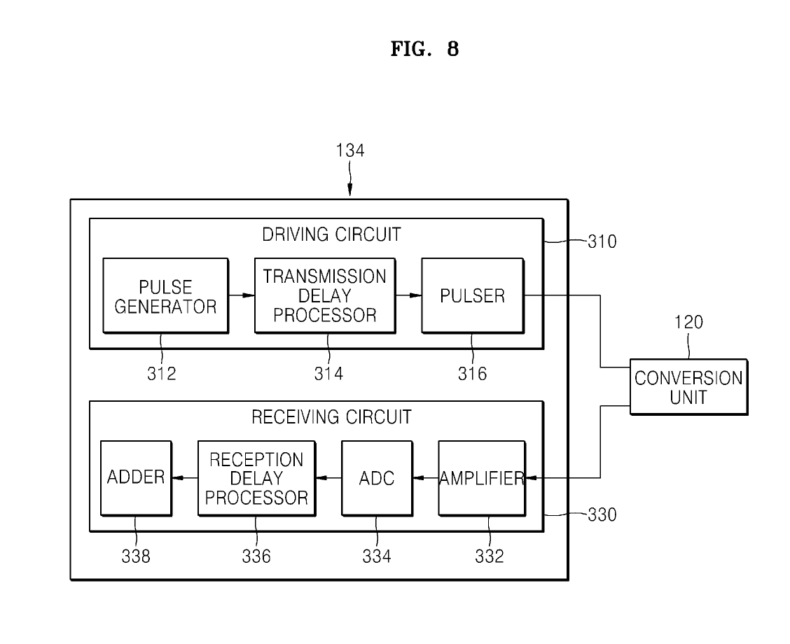

FIG. 8 is a block diagram of the second circuit unit 134 of FIG. 1, according to an exemplary embodiment. Referring to FIG. 8, the second circuit unit 134 may include a driving circuit 310 providing an electrical signal for driving the conversion unit 120, and a receiving circuit 330 receiving an electrical signal corresponding to ultrasound from the conversion unit 120 and generating ultrasound data.

The driving circuit 310 may include a pulse generator 312, a transmission delay processor 314, and a pulser 316. The pulse generator 312 generates rate pulses for generating transmission ultrasound that depends on a pulse repetition frequency (PRF). The transmission delay processor 314 applies a delay time for determining transmission directionality to the rate pulses generated by the pulse generator 312. Each of the rate pulses having the delay time applied thereto corresponds to each of the first conversion elements 210 included in the conversion unit 120. The pulser 316 applies a driving signal or a driving pulse to the first conversion elements 210 based on timing that corresponds to each of the rate pulses having the delay time applied thereto.

The receiving circuit 330 may generate the ultrasound data by processing a signal received from the conversion unit 120, and may include an amplifier 332, an analog-to-digital converter (ADC) 334, a reception delay processor 336, and an adder 338.

The amplifier 332 amplifies the signal received from the conversion unit 120, and the ADC 334 performs analog-to-digital conversion on the amplified signal. The reception delay processor 336 applies a delay time for determining reception directionality, to the digitized signal. The adder 338 generates the ultrasound data by adding signals processed by the reception delay processor 336.

The amplifier 332 of the receiving circuit 330 may apply different gains according to whether the stimulation unit 110 has stimulated an object. For example, when the conversion unit 120 receives an echo signal of ultrasound applied to a non-stimulated object, the amplifier 332 may amplify the echo signal received from the conversion unit 120 by using a large gain. When the conversion unit 120 receives an echo signal of ultrasound applied to a stimulated object, the amplifier 332 may amplify the echo signal received from the conversion unit 120 by using a small gain.

Each first conversion element 210 may be individually connected to the driving circuit 310 and the receiving circuit 330, or each group into which the first conversion elements 210 are divided may be connected to the driving circuit 310 and the receiving circuit 330. Alternatively, some of the first conversion elements 210 may be connected to the driving circuit 310, and the rest may be connected to the receiving circuit 330. The second circuit unit 134 may be, but is not limited to, an ASIC.

The ultrasonic probe 100 may use an imaging portion 152 to acquire a general ultrasonic image. For example, the driving circuit 310 of the second circuit unit 134 applies an electrical transmission signal to the conversion unit 120, which converts the electrical transmission signal into ultrasound and transmits the ultrasound to the object. The conversion unit 120 receives ultrasound reflected by the object, i.e., an ultrasound echo signal, converts the ultrasound echo signal into an electrical reception signal, and outputs the electrical reception signal to the receiving circuit 330. The receiving circuit 330 may generate image data from the electrical reception signal. The image data generated by the receiving circuit 330 is the basis of an ultrasonic image. An ultrasonic image of a non-stimulated object is referred to as a general ultrasonic image.

The stimulation unit 110 included in the ultrasonic probe 100 may give a stimulus to the object so that sound waves or shear waves are induced from the object. The stimulus may be light or pressure waves.

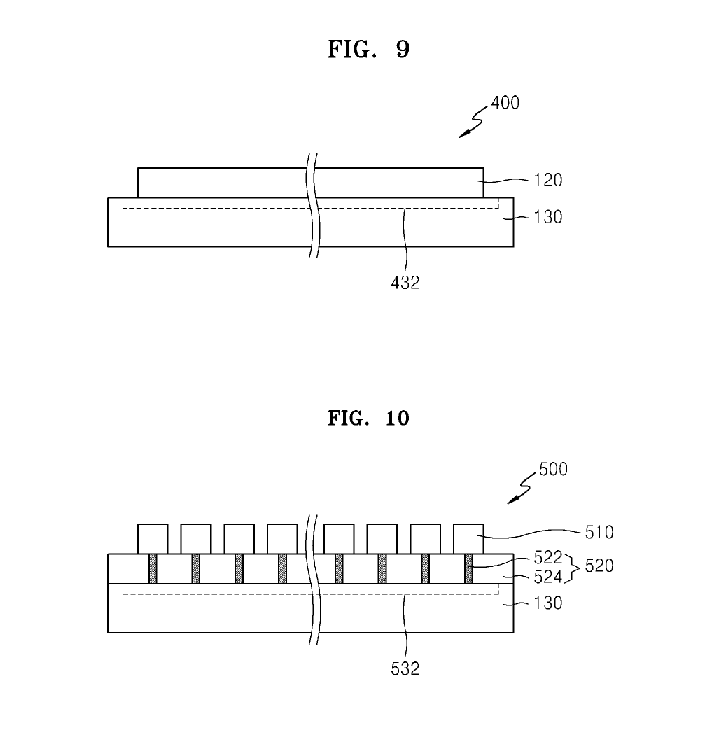

FIG. 9 illustrates a stimulation portion 400 for inducing sound waves, according to an exemplary embodiment. The stimulation portion 400 may correspond to the stimulation portion 158 of an exemplary embodiment of FIG. 1.

A stimulation unit 120 may provide light to an object so that sound waves are induced from the object. The stimulation unit 120 may include a laser diode for generating laser. The laser may be a pulse laser, and a pulse width thereof may be a nano or pico size. The induced sound waves may be ultrasound. The stimulation unit 120 may include a single laser diode or a plurality of laser diodes. A first circuit unit 432 may include a driving circuit capable of driving the laser diode and may correspond to the first circuit unit 132 of an exemplary embodiment of FIG. 1. The first circuit unit 432 may include a single driving circuit for each laser diode, or a single driving circuit for a plurality of laser diodes. Although not illustrated in FIG. 9, the stimulation unit 120 may further include a focusing unit that focuses the light generated by a plurality of laser diodes.

The laser energy emitted from the stimulation portion 400 is absorbed by tissue within the object, which causes a rapid increase in the temperature of the object and rapid thermal expansion of the object. Due to this thermal expansion, sound waves (for example, ultrasound) may be generated within the object. Since different types of tissue of an object have different light absorption characteristics, an image of the object may be obtained based on the intensity and position of sound waves generated by the object. An image obtained using light stimulation may be referred to as a photo-acoustic image.

The photo-acoustic image may have a high contrast due to light absorption and have high resolution. Therefore, the photo-acoustic image is useful for early detection of cancer. The photo-acoustic image may be obtained using the stimulation portion and the imaging portion of the ultrasonic probe 100. For example, the first circuit unit 432 applies an electrical stimulation signal to the stimulation unit 120, and the stimulation unit 120 converts the electrical stimulation signal into a stimulus, for example, light, and applies the stimulus to the object. Due to the application of the light to the object, tissue within the object is increased in temperature and thermally expanded by the light, and sound waves (for example, ultrasound) are generated. Then, the conversion unit 120 receives ultrasound reflected by the object, converts the ultrasound into an electrical reception signal, and applies the electrical reception signal to the receiving circuit 330. The receiving circuit 330 may generate image data from the electrical reception signal. The image data generated by the receiving circuit 330 is the basis of a photo-acoustic image.

Alternatively, when sound waves instead of ultrasound are generated from the object by light, an ultrasonic probe may generate ultrasound and may receive an ultrasound echo signal including information about the sound waves, for example, the speed of the sound waves.

Although the stimulation unit 120 is a laser diode in FIG. 9, exemplary embodiments are not limited thereto. The stimulation unit 120 may be any light source as long as it can generate light that induces sound waves from an object. For example, the stimulation unit 120 may include a light emitting diode (LED), black body radiator or a lamp.

FIG. 10 illustrates a stimulation portion 500 for inducing shear waves, according to an exemplary embodiment. A stimulation unit 120 may provide pressure waves to an object so that shear waves are induced from the object. The pressure waves may be the force of point impulse. The point impulse force may be ultrasound.

The stimulation unit 120 may have the same structure as that of the conversion unit 120 of FIGS. 2A and 2B, which converts an electrical signal into ultrasound. For example, the stimulation unit 120 may include a plurality of second conversion elements 510, a connection unit 520, which supports the second conversion elements 510 and electrically connects the second conversion elements 510 to the circuit board 130. The connection unit 520 may include an ultrasound-backing unit 522, which supports the second conversion elements 510 at a bottom side of the second conversion elements 510 and electrodes 524, which are disposed within the ultrasound-backing unit 522 and electrically connect the second conversion elements 510 to the first circuit unit 132. The structures of the connection unit 520, the ultrasound-backing unit 522, and electrodes 524 are similar to that described above with respect to similar elements of FIGS. 2A and 2B and, thus, repeated description is omitted.

The second conversion elements 510 may be arranged in a one-dimensional or two-dimensional manner. The reason why the second conversion elements 510 of the stimulation unit 120 are arranged one-dimensionally is to set respective voltages applied to the second conversion elements 510 to be large. Since ultrasound emitted from the second conversion elements 510 should induce shear waves from the object, the intensity of the ultrasound may be greater than that of the ultrasound emitted from the conversion unit 120. For example, a voltage applied to the stimulation unit 120 may be 150V or more. A first circuit unit 532 drives the second conversion elements 510 of the stimulation unit 120. Since the structure of the first circuit unit 532 is the same as that of the driving circuit 310 of the second circuit unit 134, a detailed description thereof will be omitted. The first circuit unit 532 only stimulates an object, and does not need to include the receiving circuit 330.

Ultrasonic energy emitted from the stimulation unit 120 is focused on tissue within the object, and the tissue generates shear waves while being restored by stress. The shear modulus of shear waves varies depending on the mechanical coefficient of tissue. For example, abnormal tissue such as cancer or a tumor may have high elasticity compared with normal tissue. Accordingly, the abnormal tissue such as cancer or a tumor may have a higher shear modulus than normal tissue around the abnormal tissue. Therefore, since the displacement of shear waves may vary according to the mechanical coefficient, for example, the elasticity coefficient, of tissue, the displacement of shear waves may be calculated from an ultrasonic image of the object in which the shear waves are travelling. An ultrasonic image including such information about shear waves may be referred to as elastography.

The elastography may be obtained using the stimulation portion and the imaging portion of the ultrasonic probe 100. For example, the first circuit unit 532 applies an electrical stimulation signal to the stimulation unit 120, and the stimulation unit 120 converts the electrical stimulation signal into a stimulus, for example, ultrasound, and applies the stimulus to the object. Then, ultrasound is focused on the object, and thus the object generates shear waves due to stress. Thereafter, the conversion unit 120 transmits ultrasound to the object and receives ultrasound reflected by the object having the shear waves flowing therein, i.e., an ultrasound echo signal. The ultrasound echo signal includes information about the shear waves. The conversion unit 120 converts the ultrasound echo signal into an electrical reception signal and transmits the electrical reception signal to the receiving circuit 330. The receiving circuit 330 may generate image data from the electrical reception signal. The image data generated by the receiving circuit 330 is the basis of the elastography.

As described above, the single ultrasonic probe 100 may obtain both a signal serving as the basis of a general ultrasonic image and a photo-acoustic image and a signal serving as the basis of a general ultrasonic image and elastography. In addition, since a circuit for driving a stimulation unit and a conversion unit and a circuit for receiving an electrical signal corresponding to ultrasound are formed within a single circuit board, a compact ultrasonic probe may be obtained.

The described-above single ultrasonic probe includes a single imaging portion and a single stimulation portion. Alternatively, a plurality of stimulation portions may be included. The plurality of stimulation portions may provide different types of stimuli to an object.

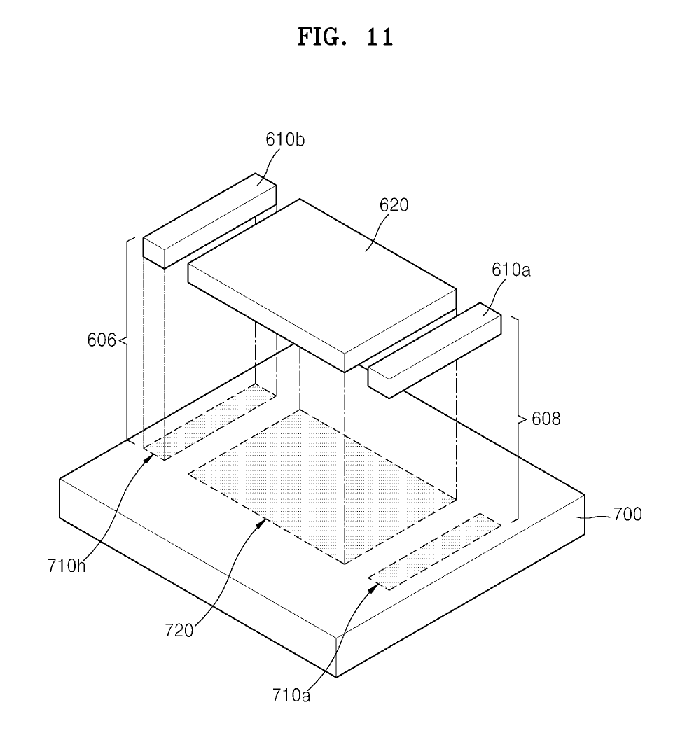

FIGS. 11 through 13 are schematic perspective views of ultrasonic probes each having a plurality of stimulation portions, according to exemplary embodiments.

As illustrated in FIG. 11, a first stimulation portion 606 may include a first stimulation unit 610a and a first second sub-circuit unit 710a. A second stimulation portion 608 may include a second stimulation unit 610b and a second sub-circuit unit 710b. The first and second stimulation units 610a and 610b may be disposed separate from each other with a conversion unit 620 interposed therebetween, and first second sub-circuit units 710a and 710b may be disposed separate from each other with a second circuit unit 720 interposed therebetween. The first and second sub-circuit units 710a and 710b and the second circuit unit 720 may be formed on a single circuit board 700, the first sub-circuit unit 710a may drive the first stimulation unit 610a, and the second sub-circuit unit 710b may drive the second stimulation unit 610b. The conversion unit 620 and the first and second stimulation units 610a and 610b may be arranged in correspondence to that of the second circuit unit 720 and the first and second sub-circuit units 710a and 710b. For example, the conversion unit 620 may be disposed on an upper surface of the second circuit unit 720, the first stimulation unit 610a may be disposed on an upper surface of the first sub-circuit unit 710a, and the second stimulation unit 610b may be disposed on an upper surface of the second sub-circuit unit 710b.

The first and second stimulation units 610a and 610b may provide stimuli of the same type to an object. For example, the first and second stimulation units 610a and 610b may be laser diodes or at least one second conversion element. Due to the above-described arrangement of the first and second stimulation units 610a and 610b on opposing sides of the conversion unit 620, stimuli emitted by the first and second stimulation units 610a and 610b may be focused on a region of interest of the object, which is located over the conversion unit 620.

The first and second sub-circuit units 710a and 710b may independently and respectively drive the first and second stimulation units 610a and 610b. For example, if the first and second stimulation units 610a and 610b include conversion elements that provide pressure waves, the frequency of ultrasound transmitted by the first stimulation unit 610a may be different from that of ultrasound transmitted by the second stimulation unit 610b. When ultrasound signals having different frequencies are focused on an object, shear waves corresponding to a difference between the two frequencies may be induced due to non-linearity of the object. Elastography may be obtained from the displacement information of the induced shear waves.

Alternatively, the first and second stimulation units 610a and 610b may provide different types of stimuli to the object. For example, the first stimulation unit 610a may provide light to the object, and the second stimulation unit 610b may provide pressure waves to the object. When a photo-acoustic image is desired to be obtained, the first sub-circuit unit 710a and the second circuit unit 720 may operate in synchronization with each other. When elastography is desired to be obtained, the second sub-circuit unit 710b and the second circuit unit 720 may operate in synchronization with each other.

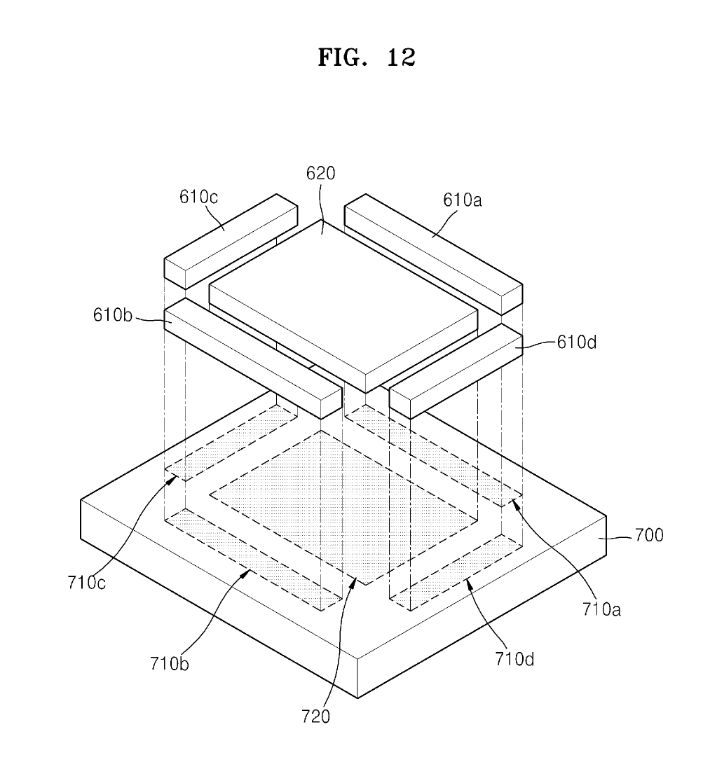

As illustrated in FIG. 12, first, second, third, and fourth stimulation units 610a, 610b, 610c, and 610d may be respectively disposed on four sides of the conversion unit 620 and may be separate from one another, and first, second, third, and fourth sub-circuit units 710a, 710b, 710c, and 710d may be disposed on four sides of the second circuit unit 720 and may be separate from one another. The second circuit unit 720 and the first, second, third, and fourth sub-circuit units 710a, 710b, 710c, and 710d may be formed on the single circuit board 700. The first, second, third, and fourth sub-circuit units 710a, 710b, 710c, and 710d may respectively drive the first, second, third, and fourth stimulation units 610a, 610b, 610c, and 610d. The first, second, third, and fourth sub-circuit units 710a, 710b, 710c, and 710d may independently drive the first, second, third, and fourth stimulation units 610a, 610b, 610c, and 610d, or at least two of the first, second, third, and fourth sub-circuit units 710a, 710b, 710c, and 710d may interoperate.

The conversion unit 620 and the first, second, third, and fourth stimulation units 610a, 610b, 610c, and 610d are arranged in correspondence to that of the second circuit unit 720 and the first, second, third, and fourth sub-circuit units 710a, 710b, 710c, and 710d. For example, the conversion unit 620 may be disposed on an upper surface of the second circuit unit 720, and the first, second, third, and fourth stimulation units 610a, 610b, 610c, and 610d may be respectively disposed on upper surfaces of the first, second, third, and fourth sub-circuit units 710a, 710b, 710c, and 710d. The first and second stimulation units 610a and 610b may face each other, and the third and fourth stimulation units 610c and 610d may face each other. At least one of the first and second stimulation units 610a and 610b may form a 2D arrangement together with at least one of the third and fourth stimulation units 610c and 610d.

The first and second stimulation units 610a and 610b may provide a first stimulus, and the third and fourth stimulation units 610c and 610d may provide a second stimulus. For example, the first and second stimulation units 610a and 610b may provide light as the first stimulus, and the third and fourth stimulation units 610c and 610d may provide pressure waves as the second stimulus. Thus, the ultrasonic probe 100 may acquire three types of images, i.e., a general ultrasound image, a photo-acoustic image, and elastography, via the single conversion unit 620. For example, a general ultrasound image may be acquired by an operation of the conversion unit 620, a photo-acoustic image may be acquired by operations of the first and second stimulation units 610a and 610b and the conversion unit 620, and elastography may be acquired by operations of the third and fourth stimulation units 610c and 610d and the conversion unit 620.

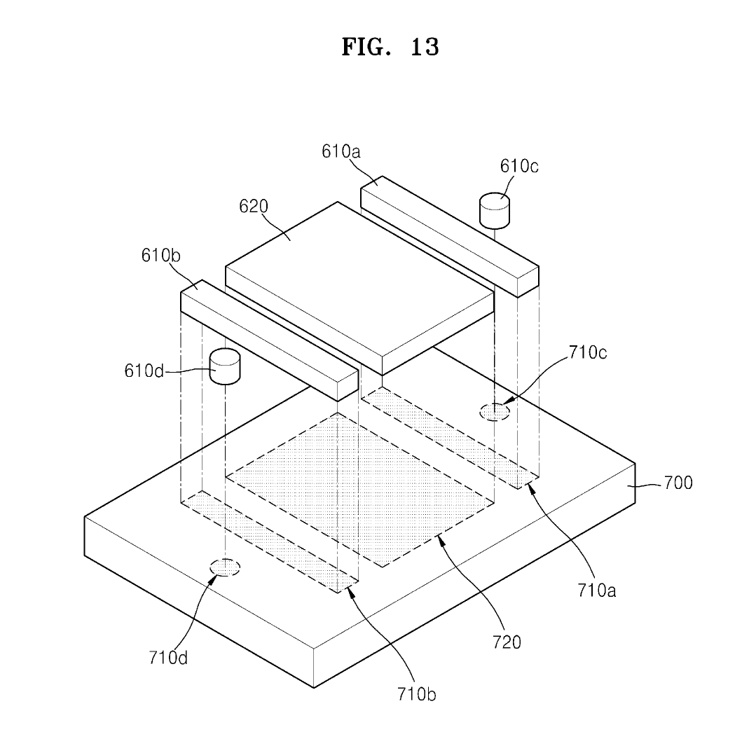

Alternatively, as illustrated in FIG. 13, first, second, third, and fourth stimulation units 610a, 610b, 610c, and 610d may be arranged one-dimensionally with the conversion unit 620 interposed therebetween, and first, second, third, and fourth sub-circuit units 710a, 710b, 710c, and 710d may be arranged one-dimensionally with the second circuit unit 720 interposed therebetween. The second circuit unit 720 and the first, second, third, and fourth sub-circuit units 710a, 710b, 710c, and 710d may be formed on the single circuit board 700. The first, second, third, and fourth sub-circuit units 710a, 710b, 710c, and 710d may respectively drive the first, second, third, and fourth stimulation units 610a, 610b, 610c, and 610d.

The conversion unit 620 and the first, second, third, and fourth stimulation units 610a, 610b, 610c, and 610d may be arranged in correspondence to that of the second circuit unit 720 and the first, second, third, and fourth sub-circuit units 710a, 710b, 710c, and 710d. For example, the conversion unit 620 may be disposed on an upper surface of the second circuit unit 720, and the first, second, third, and fourth stimulation units 610a, 610b, 610c, and 610d may be respectively disposed on upper surfaces of the first, second, third, and fourth sub-circuit units 710a, 710b, 710c, and 710d. The first and second stimulation units 610a and 610b may be disposed on the opposing sides of the conversion unit 620 facing each other, and the third and fourth stimulation units 610c and 610d may be disposed on the opposing sides of the conversion unit 620 facing each other.

The first and second stimulation units 610a and 610b may provide a first stimulus, and the third and fourth stimulation units 610c and 610d may provide a second stimulus. For example, the first and second stimulation units 610a and 610b may provide pressure waves (e.g., ultrasound) as the first stimulus, and the third and fourth stimulation units 610c and 610d may provide light as the second stimulus.

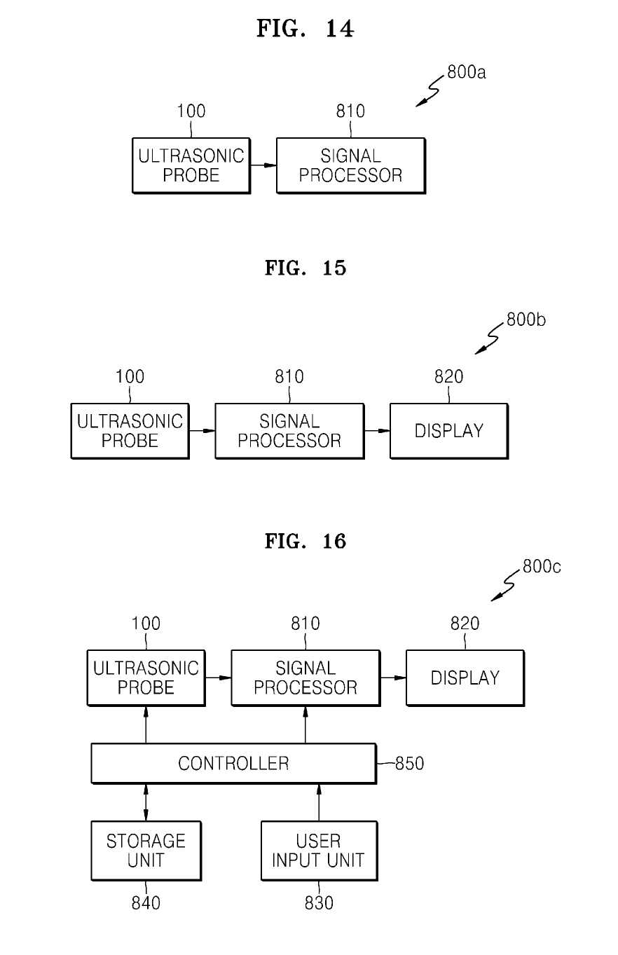

The ultrasonic probe 100 as described above may be applicable to medical apparatuses such as diagnostic apparatuses or treatment apparatuses. FIG. 14 is a block diagram of a medical apparatus 800a including the ultrasonic probe 100, according to an exemplary embodiment. Referring to FIG. 14, the medical apparatus 800a includes the ultrasonic probe 100, and a signal processor 810 for generating an ultrasonic image from ultrasound data received from the ultrasonic probe 100. The ultrasonic probe 100 has already been described above, so a detailed description thereof will be omitted.

The signal processor 810 generates the ultrasonic image by processing the ultrasound data generated by the ultrasonic probe 100. For example, the signal processor 810 may acquire the ultrasonic image by performing beamforming on the ultrasound data generated by the ultrasonic probe 100. The ultrasonic image may be at least one of an image obtained during a brightness mode (B mode) in which magnitude of an echo signal of ultrasound reflected from an object is expressed as a brightness; an image obtained during a Doppler mode in which an image of a moving object is shown as a spectrum image by using a Doppler effect; an image obtained during a motion mode (M mode) in which motion of an object according to time is shown at a constant location; an image obtained during an elasticity mode in which a difference between a case of applying compression to an object and a case of not applying the compression to the object is expressed as an image; and an image obtained during a color mode (C mode) in which a speed of a moving target object is expressed as a color by using a Doppler effect. One of currently usable methods of generating an ultrasonic image may be applied to one or more exemplary embodiments, and thus, detailed descriptions thereof are omitted here. Accordingly, in the present exemplary embodiment, the ultrasonic image may include images obtained in dimensional modes such as one-dimensional (1D), two-dimensional (2D), three-dimensional (3D), four-dimensional (4D), or the like. The ultrasonic image includes elastography.

The signal processor 810 may calculate the displacement of shear waves from elastography and may calculate a mechanical coefficient of the object from the calculated displacement of the shear waves. Thus, the signal processor 810 may further include a displacement calculator for calculating a displacement of the shear waves and a coefficient calculator for calculating the mechanical coefficient (for example, an elasticity coefficient) of the object, in addition to an image processor for acquiring the ultrasonic image.

In detail, the image processor may acquire a plurality of ultrasonic images by performing beamforming on electrical signals corresponding to an echo signal. The ultrasonic image is an ultrasonic image for a region of interest including shear waves. After shear waves are induced into the region of interest, the image processor may sequentially acquire a plurality of ultrasonic images at regular time intervals.

The displacement calculator selects one of the ultrasonic images, as a reference frame. For example, the displacement calculator may select as the reference frame an ultrasonic image acquired last from among the plurality of ultrasound images, or may select as the reference frame an ultrasonic image for a region of interest after shear waves have passed through the region of interest. The displacement calculator may more accurately calculate the displacement of shear waves by selecting the reference frame from among the plurality of ultrasonic images.

The displacement calculator may calculate the displacement of shear waves by comparing each of the plurality of ultrasonic images with the reference frame. A cross correlation technique may be used to compare each of the plurality of ultrasound images with the reference frame.

The coefficient calculator calculates the mechanical coefficient of tissue by using the displacement of the shear waves. For example, the coefficient calculator calculates the movement speed of the shear waves by using displacement components respectively corresponding to coordinate axes included in the displacement of the shear waves. The coefficient calculator may calculate a shear coefficient of the tissue by multiplying the square of the calculated movement speed by the density of the tissue. The coefficient calculator may also calculate, for example, the strength of the tissue by using the displacement of the shear waves.

FIGS. 15 and 16 are block diagrams of medical apparatuses 800b and 800c according to other exemplary embodiments. Referring to FIG. 15, the medical apparatus 800b may further include at least one display 820 in addition to the ultrasonic probe 100 and the signal processor 810. The display 820 displays information that is processed in the medical apparatus 800b. For example, the display 820 may display the ultrasonic image generated by the signal processor 810, and may also display a graphical user interface (GUI) or the like for requesting a user input.

The display 820 may include at least one of a liquid crystal display (LCD), a thin film transistor-liquid crystal display (TFT-LCD), an organic light-emitting diode (OLED), a flexible display, a 3D display, and an electrophoretic display. For example, the medical apparatus 800b may include at least two displays 820.

Alternatively, referring to FIG. 16, the medical apparatus 800c may further include at least one of a user input unit 830, a storage unit 840, and a controller 850, in addition to the ultrasonic probe 100, the signal processor 810, and the display 820. The user input unit 830 denotes a unit via which a user inputs data for controlling a medical apparatus. The user input unit 830 may include a keypad, a mouse, a touch panel, a trackball, or the like. The user input unit 830 in one or more exemplary embodiments is not limited thereto, and thus may further include various input units such as a jog wheel, a jog switch, or the like.

A touch panel may detect an actual touch and a proximate touch. With the actual touch, a pointer actually touches a screen, and, with the proximate touch, the pointer approaches the screen but does not actually touch the screen. In the present exemplary embodiment, the pointer is a tool used for actually touching or proximately touching a predetermined portion of the touch panel. Examples of the tool include a stylus pen, a part of a body such as a finger, or the like.

The touch panel and the display 820 may form a multiple-layer structure to embody a touch screen. The touch screen may be variously embodied as a capacitive touch screen, a pressure resistive touch screen, an infrared beam sensing touch screen, a surface acoustic wave touch screen, an integral strain gauge touch screen, a piezoelectric touch screen, or the like. Since the touch panel performs functions of both the display 820 and the user input unit 830, the touch panel has excellent utilization.

Although not illustrated, to detect the actual touch or the proximate touch on the touch pad, the touch pad may internally or externally have various sensors. An example of the sensor used to detect the actual touch or the proximate touch on the touch pad may include a tactile sensor. The tactile sensor detects a contact of a specific object, with a sensitivity that is equal to or greater than that of human touch. The tactile sensor may detect various types of information, such as the roughness of a contact surface, the hardness of the contact object, the temperature of a contact point, or the like.

Another example of the sensor used to detect the actual touch or the proximate touch on the touch pad may include a proximity sensor. The proximity sensor detects the existence of an object that approaches a predetermined detection surface or that exists nearby, by using a force of an electro-magnetic field or infrared rays, without using a mechanical contact. Examples of the proximity sensor include a transmission-type photoelectric sensor, a direct reflection-type photoelectric sensor, a mirror reflection-type photoelectric sensor, a high frequency oscillation-type proximity sensor, a capacity-type proximity sensor, a magnetic proximity sensor, an infrared-type proximity sensor, or the like.

The storage unit 840 stores various types of information processed in the medical apparatus 800c. For example, the storage unit 840 may store medical data, such as an image or the like, related to diagnosing an object, and may store an algorithm or program, which is performed in the medical apparatus 800c.

The storage unit 840 may include at least one of storage medium including at least one of a flash memory, a hard disk, a multimedia card micro, a card type memory (for example, a secure digital (SD) or extreme digital (XD) memory), a random access memory (RAM), a static random access memory (SRAM), a read-only memory (ROM), an electrically erasable programmable ROM (EEPROM), a programmable ROM (PROM), a magnetic memory, a magnetic disk, and an optical disk. The medical apparatus 800c may operate a web storage or a cloud server on the internet which performs a storage function of the storage unit 840.

The controller 850 controls operations of the medical apparatus 800c. That is, the controller 850 may control operations performed by the ultrasonic probe 100, the signal processor 810, the display 820, and the like shown in FIG. 16. For example, the controller 850 may control the signal processor 810 to produce an image by using a user command that is input via the user input unit 830 or by using the program stored in the storage unit 840. The controller 850 may also control the display 820 to display the image produced by the signal processor 810.

An ultrasonic probe according to an exemplary embodiment may stimulate an object via a single ultrasonic probe and acquire information about a stimulated object.

The ultrasonic probe may acquire information including the mechanical characteristics of the object.

The ultrasonic probe may be used to acquire an image having high resolution.

The ultrasonic probe may be used to adaptively acquire various ultrasonic images according to the characteristics, diagnostic purposes, and the like of an object.

A medical diagnostic apparatus according to an exemplary embodiment may acquire various types of ultrasonic images.

The foregoing exemplary embodiments and advantages are merely exemplary and are not to be construed as limiting. Descriptions of features or aspects within each embodiment should typically be considered as available for other similar features or aspects in other embodiments.

The present teaching can be readily applied to other types of apparatuses. Also, the description of the exemplary embodiments is intended to be illustrative, and not to limit the scope of the claims, and many alternatives, modifications, and variations will be apparent to those skilled in the art.

What is claimed is:

1. An ultrasonic probe comprising:
a stimulation unit which stimulates an object so that particular waves are induced;
a conversion unit which receives at least one of the particular waves and ultrasound including information about the particular waves; and
a circuit board comprising a first circuit unit that drives the stimulation unit, and a second circuit unit that receives electrical signals corresponding to at least one of the particular waves and the ultrasound, from the conversion unit.

2. The ultrasonic probe of claim 1, wherein the particular waves comprise at least one of sound waves and shear waves.

3. The ultrasonic probe of claim 1, wherein information about the particular waves comprises at least one of a displacement, a speed, and an intensity of the particular waves.

4. The ultrasonic probe of claim 1, wherein the stimulation unit and the conversion unit are arranged in correspondence to that of the first circuit unit and the second circuit unit.

5. The ultrasonic probe of claim 4, wherein the stimulation unit is disposed on an upper surface of the first circuit unit, and
the conversion unit is disposed on an upper surface of the second circuit unit.

6. The ultrasonic probe of claim 1, wherein the conversion unit comprises:
first conversion elements that are arranged two-dimensionally and convert the ultrasound into the electrical signals.

7. The ultrasonic probe of claim 1, wherein at least one of the first circuit unit and the second circuit unit comprises an application specific integrated circuit (ASIC).

8. The ultrasonic probe of claim 1, wherein the stimulation unit provides light configured to induce sound waves from the object.

9. The ultrasonic probe of claim 8, wherein the light comprises a pulse laser.

10. The ultrasonic probe of claim 8, wherein the stimulation unit comprises a laser diode.

11. The ultrasonic probe of claim 1, wherein the second circuit unit comprises a driving circuit that drives the conversion unit.

12. The ultrasonic probe of claim 1, wherein the stimulation unit comprises first and second stimulation units that are disposed separated from each other with the conversion unit interposed therebetween, and
the first circuit unit comprises a first sub-circuit unit that drives the first stimulation unit and a second sub-circuit unit that drives the second stimulation unit.

13. The ultrasonic probe of claim 12, wherein the first and second sub-circuit units independently drive the first and second stimulation units, respectively.

14. The ultrasonic probe of claim 12, wherein the first and second stimulation units provide pressure waves configured to induce shear waves from the object.

15. The ultrasonic probe of claim 14, wherein the pressure waves comprise ultrasound.

16. The ultrasonic probe of claim 15, wherein a frequency of the ultrasound transmitted by the first stimulation unit is different from that of the ultrasound transmitted by the second stimulation unit.

17. The ultrasonic probe of claim 12, wherein each of the first and second stimulation units comprises second conversion elements that convert electrical signals into the ultrasound.

18. The ultrasonic probe of claim 17, wherein the second conversion elements are arranged one-dimensionally, in the first stimulation unit and the second stimulation unit.

19. The ultrasonic probe of claim 12, wherein one of the first and second stimulation units provides light, and other one of the first and second stimulation units provides pressure waves.

20. The ultrasonic probe of claim 12, wherein the stimulation unit further comprises third and fourth stimulation units that are disposed separated from each other with the conversion unit interposed therebetween, and
the first circuit unit further comprises a third sub-circuit unit that drives the third stimulation unit and a fourth sub-circuit unit that drives the fourth stimulation unit.

21. The ultrasonic probe of claim 20, wherein at least one of the first and second stimulation units is arranged together with at least one of the third and fourth stimulation units as a two-dimensional (2D) array.

22. The ultrasonic probe of claim 20, wherein the first and second stimulation units provide a first stimulus, and
the third and fourth stimulation units provide a second stimulus.

23. An ultrasonic diagnosing apparatus comprising:
the ultrasonic probe of claim 1; and
a signal processor that processes a signal received from the ultrasonic probe to generate an image.

24. The ultrasonic diagnosing apparatus of claim 23, further comprising a display that displays the image.
